(19) United States
(12) Patent Application Publication
(10) Pub. No.: US 2019/0000905 A1
(43) Pub. Date: Jan. 3, 2019

(54) Gynostemma pentaphyllum fermented extracts comprising Saponins as an active functional food ingredient and preparation method thereof

(71) Applicant: BTC Co., Ltd., Gwangmyeong-si (KR)

(72) Inventors: Joo-Myung MOON, Ansan-si (KR); Tea Young KIM, Ansan-si (KR); Myoung Seo KU, Ansan-si (KR)

(21) Appl. No.: 15/636,308

(22) Filed: Jun. 28, 2017

(51) Int. Cl. A61K 36/424; A61K 8/9789; A61Q 19/00; A61K 31/704; A61K 8/63; A23L 33/105

(52) U.S. Cl. CPC A61K 36/424; A61K 8/9789; A61Q 19/00; A61K 31/704; A61K 8/63; A23V 2002/00; A61K 2236/19; A61K 2236/331; A61K 2236/15; A61K 2800/85; A23L 33/105

(57) ABSTRACT

A method for preparing an extract of fermented Gynostemma pentaphyllum with increased contents of saponins or low-molecular-weight saponins, which includes a step of preparing a Gynostemma pentaphyllum fermentation product by fermenting Gynostemma pentaphyllum with a microorganism selected from Aspergillus oryzae, Aspergillus awamori, Aspergillus kawachii, Lactobacillus brevis and Lactobacillus plantarum; and a step of extracting the Gynostemma pentaphyllum fermentation product with hot water.

### TECHNICAL FIELD

The present disclosure relates to an extract of fermented Gynostemma pentaphyllum with increased contents of low-molecular-weight saponins, a method for preparing the same and a use of the same. More specifically, it relates to a method for increasing the contents of total saponins and low-molecular-weight saponins by extracting Gynostemma pentaphyllum fermented with microorganisms with hot water and a use of the extract of fermented Gynostemma pentaphyllum prepared thereby.

### BACKGROUND

Dolwoe (Gynostemma pentaphyllum) also known as dungkulcha is a dicotyledon belonging to the family Cucurbitaceae of the order Cucurbitales. It is a perennial vine. It is distributed mainly in humid areas, e.g., seashore, riverside, etc., of Manchuria, Taiwan, Japan, Malaysia, India, etc. In Korea, it grows wildly or is cultivated in the fields and mountains of the southern part, Jeju Island and Ullenung Island.

The Pharmaceutical Society of Japan reported that Gynostemma pentaphyllum contains more saponin ingredients than ginseng. Also in Korea, studies have been actively conducted about Gynostemma pentaphyllum saponins and the functions of Gynostemma pentaphyllum extracts and it was reported that the Gynostemma pentaphyllum extract has anti-hyperlipidemic, anti-diabetic, anti-inflammatory, anti-ulcer and body fat-reducing effects [(a) Journal of Ginseng Research 8(2) 1984, 172-177; (b) Korean Patent Registration No. 0930580; (c) Korean Patent Registration No. 1450571].

Gynostemma pentaphyllum contains dammarane-type saponins including various gypenosides as well as other glycosides such as gynosaponins, primeverosides, bisdesmosides, sophorosides, gentiobiosides, rutinosides, etc., steroids, sugars, etc., depending on the regions where it grows. The diverse Gynostemma pentaphyllum saponins are known to exhibit various health-improving effects such as improvement of lipid metabolism, defense against cardiovascular diseases, lowering of blood sugar, advantageous effect on the central nervous system, anti-cancer effect, inhibition of platelet aggregation, tonic effect, etc. It is reported that these effects overlap considerably with those of the ginsenosides of Korean ginseng (Panax ginseng C. A. Meyer) as active ingredients. It is because ginseng and Gynostemma pentaphyllum both contain dammarane-type saponins [Chemistry and pharmacology of Gynostemma pentaphyllum. Phytochemistry Reviews, 4: 2005, 197-219].

It is known that Gynostemma pentaphyllum is effective in reducing superoxide anions through its antioxidant activity, protecting endothelial cells, improving cardiovascular functions, reducing cholesterols, regulating immune functions, inhibiting cancer cell growth, improving diabetes, protecting liver functions, improving inflammations (chronic bronchitis and gastric ulcer), etc. and it is reported that Gynostemma pentaphyllum saponins facilitate fat degradation and inhibit fat synthesis by increasing the activity of AMP-activated protein kinase (AMPK, cellular energy sensor) in muscle cells and fat cells and exhibit body fat-reducing effect by increasing the fat-degrading (beta-oxidation) activity in mitochondria when treated to liver cells.

In addition, it is reported that a Gynostemma pentaphyllum extract exhibits blood sugar-regulating effect through increased sugar inflow into cells when threated to muscle cells, exhibits an effect of improving insulin resistance by inhibiting the activity of IKK-β and JNK which diminish insulin signaling when threated to muscle cells, reduces corticosterone which is a stress hormone in blood and improves the symptoms of anxiety and depression by preventing the reduction of the neurotransmitters dopamine and serotonin in the brain [Korean Journal of Pharmacognosy, Vol. 42, No. 1, pp. 32-37]. Also, it has been found out that an ethanol extract of Gynostemma pentaphyllum leaf is helpful in improving the symptoms of patients with Parkinson's disease by protecting dopaminergic neurons.

The Gynostemma pentaphyllum extract is reported to restore splenocyte proliferation decreased by oral administration of cadmium. Also, it was found out that the polysaccharide components of Gynostemma pentaphyllum promote the secretion of nitric oxide by activating peritoneal macrophages and also promote the secretion of TNF-α in a concentration-dependent manner.

Some saponins can be absorbed only after they are enzymatically converted to active ingredients in the human body. Gynostemma pentaphyllum exhibits low bioavailability because most of its sugars are modified. Its effect can be maximized when bio-converted metabolites are used. Therefore, a new method for increasing the contents of low-molecular-weight saponins which are useful biologically active ingredients of the Gynostemma pentaphyllum extract is necessary in order to maximize the effects of superior health-improving substances of Gynostemma pentaphyllum.

### SUMMARY

The present disclosure is directed to providing an extract of fermented Gynostemma pentaphyllum with increased contents of saponins or low-molecular-weight saponins and a method for preparing the same.

The present disclosure is also directed to providing a health functional food or a pharmaceutical composition containing an extract of fermented Gynostemma pentaphyllum with increased contents of saponins as an active ingredient, which exhibits an effect of improving or treating stress-induced decline in immune function, obesity, diabetes or hyperlipidemia.

The present disclosure is also directed to providing a cosmetic composition containing an extract of fermented Gynostemma pentaphyllum with increased contents of low-molecular-weight saponins as an active ingredient, which exhibits an effect of whitening skin or improving wrinkles.

In an aspect, the present disclosure provides a method for preparing an extract of fermented Gynostemma pentaphyllum with increased contents of saponins or low-molecular-weight saponins, which includes a step of preparing a Gynostemma pentaphyllum fermentation product by fermenting Gynostemma pentaphyllum with a microorganism selected from Aspergillus oryzae, Aspergillus awamori, Aspergillus kawachii, Lactobacillus brevis M2 and Lactobacillus plantarum P2; and a step of extracting the Gynostemma pentaphyllum fermentation product with hot water.

In the present disclosure, the saponin may be one or more selected from dammarane-type saponin, gynosaponin, ginsenoside, primeveroside, sweroside, bisdesmoside, gentiobioside and rutinoside and includes a low-molecular-weight saponin.

In the present disclosure, the low-molecular-weight saponin may be specifically one or more selected from Rg3, Rg5, Rk1, Rh2, gypenoside L and gypenoside LI.

In the present disclosure, the fermentation may be performed at 25-45° C. for 6-240 hours.

In the present disclosure, the method may further include a step of preparing an extract of fermented Gynostemma pentaphyllum pomace by extracting fermented Gynostemma pentaphyllum pomace, which is a hot water-extracted Gynostemma pentaphyllum fermentation product, with water or a 20-70% C₁₋₄ alcohol aqueous solution at 30-130° C.; and a step of mixing the extract of fermented Gynostemma pentaphyllum pomace with the hot water extract of the Gynostemma pentaphyllum fermentation product of the step (1).

In the present disclosure, the method may further include, before fermenting the Gynostemma pentaphyllum, a pretreatment step of ultrasonicating the same with 15-25 kHz and 500-800 watt for 1-20 minutes.

In another aspect, the present disclosure provides a use of a hot water extract of Gynostemma pentaphyllum fermented with a microorganism selected from Aspergillus oryzae, Aspergillus awamori, Aspergillus kawachii, Lactobacillus brevis M2 and Lactobacillus plantarum P2 (hereinafter, ‘extract of fermented Gynostemma pentaphyllum’) prepared by the method described above.

The extract of fermented Gynostemma pentaphyllum may contain 5-85 mg/g of gypenoside L and 4-70 mg/g of gypenoside LI.

Because the extract of fermented Gynostemma pentaphyllum according to the present disclosure exhibits an effect of improving or treating obesity, diabetes, hyperlipidemia and stress-induced decline in immune function, it can be used as a pharmaceutical composition or a health functional food for preventing or treating them.

Also, the extract of fermented Gynostemma pentaphyllum according to the present disclosure can be used as a cosmetic composition because it exhibits an effect of whitening skin or improving wrinkles.

The present disclosure provides a method for preparing an extract of fermented Gynostemma pentaphyllum with increased contents of total saponins, which are useful biologically active ingredients, as compared to the existing method of preparing a Gynostemma pentaphyllum extract, in particular, with increased contents of low-molecular-weight saponins, which can be absorbed easily by the body and thus exhibit superior bioavailability, and gypenosides L and LI, which exhibit superior activity of activating AMPK (AMP-activated protein kinase). In particular, the present disclosure provides a method for preparing an extract with increased contents of low-molecular-weight saponins which are hardly found or exist in trace amounts in raw Gynostemma pentaphyllum, specifically gypenosides L and LI and ginsenosides Rg3, Rg5, Rk1, etc. The extract of fermented Gynostemma pentaphyllum according to the present disclosure exhibits an effect of improving or treating diabetes, hyperlipidemia and stress-induced decline in immune function as well as a superior cosmetic effect of whitening skin, improving wrinkles, etc.

### BRIEF DESCRIPTION OF THE DRAWINGS

FIG. 1 shows a result of investigating the effect of low-molecular-weight saponins derived from an extract of fermented Gynostemma pentaphyllum according to an exemplary embodiment of the present disclosure on the activities of AMPK, ACC and FAS in 3T3-L1 cells.

FIG. 2 shows a result of investigating the effect of low-molecular-weight saponins derived from an extract of fermented Gynostemma pentaphyllum according to an exemplary embodiment of the present disclosure on the activities of AMPK, ACC and FAS in 3T3-L1 cells.

FIG. 3 shows a result of investigating the effect of low-molecular-weight saponins derived from an extract of fermented Gynostemma pentaphyllum according to an exemplary embodiment of the present disclosure on inhibition of fat accumulation.

### DETAILED DESCRIPTION OF EMBODIMENTS

Hereinafter, the present disclosure is described in more detail.

In order to provide an extract of fermented Gynostemma pentaphyllum with increased contents of total saponins, which are useful biologically active ingredients, in particular, with increased contents of low-molecular-weight saponins, which can be absorbed easily by the body, the present disclosure provides a method for preparing an extract of fermented Gynostemma pentaphyllum with increased contents of saponins or low-molecular-weight saponins, which includes (1) a step of preparing a Gynostemma pentaphyllum fermentation product by fermenting Gynostemma pentaphyllum with a microorganism selected from Aspergillus oryzae, Aspergillus awamori, Aspergillus kawachii, Lactobacillus brevis M2 and Lactobacillus plantarum P2; and (2) a step of extracting the Gynostemma pentaphyllum fermentation product with hot water.

In the present disclosure, the Gynostemma pentaphyllum includes the areal and underground parts of the plant in the genus Gynostemma and processed products thereof.

In the present disclosure, the saponin contained in the extract of fermented

Gynostemma pentaphyllum prepared by the method may be one or more selected from dammarane-type saponin, gynosaponin, primeveroside, sweroside, bisdesmoside, gentiobioside and rutinoside. The dammarane-type saponin according to the present disclosure may include gypenoside.

The saponin contained in the Gynostemma pentaphyllum extract may include, for example, ginsenosides Rb1, Rb2, Rc, Rd, Rg3, Rg5, Rk1, Rh, etc., gypenosides V, IX, XVI, XXXVI, LXXIII, LXVII, LXX, L, LI, etc., gycomoside IV, damulin A, pectolinarin, protopanaxadiol, hydroxysaikosaponin d, majoroside F5, panicolin, canosifloside VI, etc., although not being limited thereto.

In particular, the extract of fermented Gynostemma pentaphyllum prepared according to the present disclosure has greatly increased contents of ginsenosides Rg3 and Rg5, which are biologically active substances that can be absorbed easily by the body, and gypenoside L and gypenoside LI isolated from Gynostemma pentaphyllum.

First, in order to obtain an extract with increased contents of low-molecular-weight saponins in the form of Rg3 and Rg5 and gypenosides L and LI, Gynostemma pentaphyllum is fermented with a microorganism selected from Aspergillus oryzae, Aspergillus awamori, Aspergillus kawachii, Lactobacillus brevis M2 and Lactobacillus plantarum P2. Specifically, the fermentation may be performed at 25-45° C. for 6-240 hours.

Then, after extraction, the obtained extract may be filtered to remove impurities and then concentrated. Depending on use, it can be dried or prepared into powder.

In the present disclosure, the method may further include a step of preparing an extract of fermented Gynostemma pentaphyllum pomace by extracting fermented Gynostemma pentaphyllum pomace, which is a hot water-extracted Gynostemma pentaphyllum fermentation product, with water or a 20-70% C1-4 alcohol aqueous solution at 30-130° C.; and a step of mixing the extract of fermented Gynostemma pentaphyllum pomace with the hot water extract of the Gynostemma pentaphyllum fermentation product of the step (1).

Because saponins remain in the pomace remaining after the hot water extraction of the Gynostemma pentaphyllum fermentation product, ingredients useful for the human body, specifically saponins or low-molecular-weight saponins, can be extracted from the pomace using water or a 20-70% C₁₋₄ alcohol aqueous solution. Through this step, the contents of total saponins and low-molecular-weight saponins in the finally obtained extract of fermented Gynostemma pentaphyllum can be further increased.

In an exemplary embodiment of the present disclosure, the Gynostemma pentaphyllum may be used as harvested or may be ground to further increase the contents of ginsenosides in the Gynostemma pentaphyllum extract. It may also be dried and then prepared into powder. When the Gynostemma pentaphyllum is ground or prepared into powder, the efficiency of fermentation and extraction is improved advantageously because the surface area is increased.

In the present disclosure, the method may further include, before fermenting the Gynostemma pentaphyllum with a microorganism, a step of ultrasonicating the same. In an exemplary embodiment of the present disclosure, when the Gynostemma pentaphyllum is fermented with a microorganism after ultrasonicating the same, the contents of total saponins are significantly increased. In particular, the contents of low-molecular-weight ginsenosides such as Rg3, etc. and the contents of gypenosides L and LI are greatly increased.

The inventors of the present disclosure have also found out through experiments that the contents of total saponins are further increased when the Gynostemma pentaphyllum is ultrasonicated before fermenting with the microorganism as compared to when it is ultrasonicated after the fermentation. In the present disclosure, the ultrasonic radiation may be performed with 15-25 kHz and 500-800 watt for 1-20 minutes. When the energy and time of the ultrasonic radiation are lower and shorter than the above ranges, the contents of total saponins are hardly increased. And, when they exceed the above ranges, the contents of total saponins in the extract are increased only slightly or they may be even decreased undesirably.

In the present disclosure, the C₁₋₄ alcohol used in the extraction may be one or more selected from methanol, ethanol, propanol, butanol and isopropanol. Specifically, it may be ethanol.

In an exemplary embodiment of the present disclosure, the contents of gypenoside L, gypenoside LI and ginsenosides Rg3, Rg5, etc. are increased when the Gynostemma pentaphyllum is hot water-extracted after fermentation as compared to when it is hot water-extracted without fermentation. In particular, the contents of gypenoside L and gypenoside LI which are re hardly found or exist in trace amounts in raw Gynostemma pentaphyllum are increased significantly.

In an exemplary embodiment of the present disclosure, the contents of total saponins are increased and, in particular, an extract with significantly increased contents of low-molecular-weight saponins such as Rg3, gypenosides L, LI, etc. can be obtained when the extraction is performed at 90-125° C., specifically at 110-121° C., for 1-8 hours.

In an exemplary embodiment of the present disclosure, an extract of fermented Gynostemma pentaphyllum with increased contents of total saponins can be obtained when the Gynostemma pentaphyllum is extracted after fermenting with a microorganism in the genus Aspergillus or the genus Lactobacillus as compared to when it is hot water-extracted without fermentation. In particular, an extract with increased contents of biologically useful low-molecular-weight saponins such as Rg3, gypenoside L and gypenoside LI can be obtained.

Specifically, the Gynostemma pentaphyllum may be sterilized before fermenting with the microorganism. The sterilization can be performed by any method commonly used in the art. For example, high-pressure sterilization, high-temperature sterilization or low-temperature sterilization may be employed, although not being limited thereto.

The extract of the present disclosure includes not only the extract obtained using the extraction solvent described above but also an extract which has been further purified by a commonly employed method. For example, fractions obtained by various purification methods such as separation using an ultrafiltration membrane with a predetermined molecular weight cut-off, separation by chromatography (based on size, ion, hydrophobicity or solvent affinity) are included in the scope of the extract of the present disclosure.

The ‘Gynostemma pentaphyllum extract’ obtained by fermenting the Gynostemma pentaphyllum with a microorganism in the genus Aspergillus or the genus Lactobacillus and extracting the same according to the present disclosure was found to exhibit an effect of inhibiting melanin synthesis and, at the same time, promoting collagen synthesis. Specifically, the melanin synthesis inhibition activity is increased by 40% or more, the collagen synthesis is promoted by 30% or more and cytotoxicity is decreased at the same time as compared to an extract obtained from unfermented Gynostemma pentaphyllum. Accordingly, the Gynostemma pentaphyllum extract can be used as an active ingredient of a cosmetic composition for whitening skin and improving wrinkles.

The ‘Gynostemma pentaphyllum extract’ obtained by fermenting the Gynostemma pentaphyllum with a microorganism in the genus Aspergillus or the genus Lactobacillus and extracting the same according to the present disclosure was found to exhibit an effect of restoring stress-induced decline in immune function. Specifically, it was found in a mouse model in which declined immune function was induced by administering dexamethasone that oral administration of the Gynostemma pentaphyllum extract according to the present disclosure restores cytokine production ability. Accordingly, the Gynostemma pentaphyllum extract according to the present disclosure can be used as a health-improving functional food or a pharmaceutical composition for restoring immune function.

When the Gynostemma pentaphyllum extract according to the present disclosure is used as a health functional food for enhancing immune function, the type of the health functional food is not specially limited. For example, it may be prepared into a powder, a granule, a tablet, a capsule or a drink containing the Gynostemma pentaphyllum extract as an active ingredient, although not being limited thereto. The mixing amount of the active ingredient may be determined adequately depending on purposes, e.g., prevention, health improvement, treatment, etc.

When the Gynostemma pentaphyllum extract according to the present disclosure is used as a pharmaceutical composition for enhancing immune function, the type of the pharmaceutical composition may be prepared into a formulation for oral administration such as a powder, a granule, a tablet, a capsule, a suspension, an emulsion, a syrup, an aerosol, etc. or a formulation for parenteral administration such as a formulation for external application, a suppository or a sterile solution for injection. The mixing amount of the active ingredient may be determined adequately depending on purposes, e.g., prevention, health improvement, treatment, etc.

When the Gynostemma pentaphyllum extract according to the present disclosure is prepared into a powder, a granule, a tablet or a capsule, it may further contain an adequate carrier, excipient or diluent commonly used in preparing foods. As the carrier, excipient or diluent, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate or mineral oil may be used.

For the formulation, a commonly used diluent or excipient such as a filler, an extender, a binder, a wetting agent, a disintegrant, a surfactant, etc. is used.

A solid formulation for oral administration may be prepared by mixing the extract with one or more excipient, e.g., starch, calcium carbonate, sucrose, lactose, gelatin, etc. In addition to the simple excipient, a lubricant such as magnesium stearate and talc may also be used.

A liquid formulation for oral administration may be a suspension, a solution for internal use, an emulsion, a syrup, etc. It may contain, in addition to the commonly used simple diluent such as water and liquid paraffin, various excipients, e.g., a wetting agent, a sweetener, an aromatic, a preservative, etc.

The health functional food or pharmaceutical composition containing the Gynostemma pentaphyllum extract according to the present disclosure as an active ingredient may contain various nutrients, vitamins, electrolytes, flavors, colorants, pectic acid and its salts, alginic acid and its salts, organic acids, protective colloidal thickeners, pH control agents, stabilizers, antiseptics, glycerin, alcohols and carbonating agents used in carbonated beverages. These ingredients may be used either alone or in combination. Although the mixing ratio of these additives is not limited, they are usually contained in an amount of 0.01-0.1 wt % based on 100 wt % of the composition of the present disclosure.

Hereinafter, the present disclosure will be described in detail through examples. However, the following examples are for illustrative purposes only and it will be apparent to those of ordinary skill in the art that the scope of the present disclosure is not limited by the examples.

### EXAMPLES

### Example 1

A. Fermentation

Gynostemma pentaphyllum leaf was washed, dried and then ground. After adding water to 100 g of the ground Gynostemma pentaphyllum leaf in a flask such that the water content was 50% and then sterilizing in an autoclave, the flask was allowed to cool on a clean bench. Then, the ground Gynostemma pentaphyllum leaf was fermented at 28° C. for 7 days by inoculating 5 g of Aspergillus oryzae.

B. Extraction

The fermented Gynostemma pentaphyllum leaf was extracted with hot water at 121° C. for 6 hours and then separated into a hot water extract of fermented Gynostemma pentaphyllum and a pomace by filtering the same. The pomace was extracted again with a 70 vol % ethanol aqueous solution and an extract of fermented Gynostemma pentaphyllum pomace was obtained by filtering the same. The total saponin content and the kinds and contents of respective saponins were measured for each extract. A mixture obtained by mixing the extracts was named as an extract of fermented Gynostemma pentaphyllum.

### Example 2

A Gynostemma pentaphyllum extract was prepared in the same manner as in Example 1 except that Aspergillus awamori was used instead of Aspergillus oryzae.

### Example 3

A Gynostemma pentaphyllum extract was prepared in the same manner as in Example 1 except that Aspergillus kawachii was used instead of Aspergillus oryzae.

### Example 4

A Gynostemma pentaphyllum extract was prepared in the same manner as in Example 1 except that Lactobacillus brevis M2-was used instead of Aspergillus oryzae.

### Example 5

A Gynostemma pentaphyllum extract was prepared in the same manner as in Example 1 except that Lactobacillus plantarum P2 was used instead of Aspergillus oryzae.

### Comparative Example 1

A Gynostemma pentaphyllum extract was prepared in the same manner as in Example 1 using unfermented Gynostemma pentaphyllum leaf.

### Comparative Example 2

A Gynostemma pentaphyllum extract was prepared by extracting unfermented Gynostemma pentaphyllum leaf at 80° C. for 6 hours.

### Test Example 1

### Comparison of Contents of Dam Marane-Type Saponins in Gynostemma pentaphyllum Leaves Harvested at Different Regions

In order to compare the contents of saponins in Gynostemma pentaphyllum leaves harvested at different regions, Gynostemma pentaphyllum leaves were purchased from the Guangxi Province (A) and the Fujian Province (B) of China and from Geochang (C), Yeosu (D) and Jeju (D) of Korea. Gynostemma pentaphyllum originating from Ullenung Island (F) was acquired from the Plant Extract Bank of Korea.

Samples were prepared by extracting the Gynostemma pentaphyllum with hot water at 110° C. for 12 hours.

HPLC Analysis

The HPLC analysis conditions were established such that 11 standard substances (GP L, GP LI, damulin A, ginsenosides Rb1, Rb2, Rc, Rd, Rg3, Rg5, Rk1 and Rh2) can be analyzed at the same time based on the analysis method determined by the inventors of the present disclosure based on 3 marker substances (damulin A, GP L, GP LI) in consideration of column types and separation patterns of various mobile phases. The HPLC analysis results for the 11 standard substances are shown in Table 1.

The analysis was performed using the HPLC Infinity 1260 system (Agilent) and the analysis condition was as follows. An XSelect HSS T3 column (100×2.1 mm, 2.5 μm, Waters, USA) was used. The sample injection volume was 2 μL and detection was made at 204 nm using a variable wavelength detector (VWD). As a mobile phase A, 1 mL of formic acid added to 999 mL of triply distilled ultrapure water was used. As a mobile phase B, 1 mL of formic acid added to 999 mL of acetonitrile was used. The concentration of the mobile phase B solution was maintained at 20% from 0 to 10 minutes, slowly increased to 29% until 39 minutes, to 41% 67 minutes, to 47% 70 minutes and then to 71% 90 minutes. Then, the concentration was decreased to 20% until 95 minutes and then maintained until 115 minutes. The solvent flow rate was maintained constant at 0.3 mL/min.

TABLE 1

| | Curve | R² |
|---|---|---|
| Rb1 | y = 0.4822 x + 1.762 | 0.9997 |
| Rc | y = 0.5990 x − 7.6773 | 0.9999 |
| Rb2 | y = 0.5316 x + 6.9914 | 0.9999 |
| Rd | y = 0.6117 x − 1.0448 | 0.9999 |
| GP L | y = 3.3168 x − 198.06 | 0.9999 |
| GP LI | y = 0.6805 x + 2.0813 | 0.9986 |
| Rg3 | y = 0.7422 x + 18.638 | 0.9999 |
| Da A | y = 4.6718 x + 3.3803 | 0.9999 |
| Rg5 | y = 0.5652 x + 5.3952 | 0.9999 |
| Rk1 | y = 1.1039 x + 10.934 | 0.9999 |
| Rh2 | y = 0.9891 x + 5.8976 | 0.9999 |

Q-TOF Analysis

In order to analyze the ingredients of the Gynostemma pentaphyllum leaf extracts from different regions, the 11 standard substances were analyzed by Q-TOF. Ingredients other than the standard substances were identified using the TCM (Traditional China Medicine) Library (2014, Agilent, USA) program. The Q-TOF analysis condition was as follows.

Drying gas (N2) flow rate: 17 L/min.

Drying gas (N2) temperature: 225° C.

Nebulizer pressure: 45 psi.

Sheath gas temperature: 350° C.

Sheath gas flow rate: 11 L/min.

Capillary voltage: 3,500 V.

Nozzle voltage: 2,000 V.

Fragmentor voltage: 400 V.

Skimmer voltage: 65 V.

OCT 1 RF Vₚₚ: 750 V.

Collision energy: 25 V.

Ion polarity: negative.

The result of analyzing the contents of saponins in Gynostemma pentaphyllum harvested at different regions based on the results of HPCL, Q-TOF and TCM Library is shown in Table 2. The kinds and contents of saponins were different from regions to regions. For the Gynostemma pentaphyllum originating from China, the Gynostemma pentaphyllum of the Guangxi Province contained 2 times more saponins than the Gynostemma pentaphyllum of the Fujian Province. For the Gynostemma pentaphyllum originating from Korea, the Gynostemma pentaphyllum of Jeju showed the highest saponin contents, followed by Yeosu, Geochang and Ullenung Island.

TABLE 2
Saponin contents (mg/mL)

| Region | A | B | C | D | E | F |
|---|---|---|---|---|---|---|
| Rb1 | 0.18 | 0.23 | 0.51 | 1.41 | 5.27 | 0.52 |
| Rc | 0.14 | 3.76 | 1.94 | 0.13 | 0.66 | <0.01 |
| Rd | 0.29 | 1.17 | 4.10 | 8.02 | 8.93 | 1.15 |
| GP L | 1.55 | 0.15 | 0.53 | 0.13 | 1.96 | <0.01 |
| GP LI | 0.21 | 0.21 | 0.32 | 0.22 | 0.52 | <0.01 |
| Rg3 | 5.60 | 0.39 | 0.56 | 0.80 | 1.14 | <0.01 |
| Da A | 0.01 | 0.04 | 0.26 | 0.02 | 0.80 | <0.01 |
| Rg5 | 0.05 | 0.95 | 1.05 | 0.46 | 0.90 | <0.01 |
| Rk1 | 7.87 | 0.19 | 0.71 | 0.45 | 0.54 | <0.01 |
| Rh2 | 0.30 | 0.07 | 0.05 | 0.04 | 0.03 | <0.01 |
| Total | 16.20 | 7.15 | 10.04 | 16.68 | 20.74 | 1.67 |

Therefore, it was found out that kinds and contents of saponins are different from regions to regions.

### Test Example 2

Effect of Extraction Time

In order to compare the change in the ingredients and the total saponin content of the Gynostemma pentaphyllum extract depending on extraction time, unfermented Gynostemma pentaphyllum was extracted at 121° C. for 1, 2, 3, 4, 5, 6, 7, 8, 9, 10 and 24 hours and the detected saponin ingredients and their contents were compared. It was investigated how many were detected among 3 standard substances (gypenoside L, gypenoside LI, ginsenoside Rg3).

TABLE 3

| | | Gypenoside L | Gypenoside LI | Rg3 |
|---|---|---|---|---|
| Extraction time | 1 | 6.43 | 5.48 | 1.12 |
| (hr) | 2 | 8.44 | 7.43 | 3.81 |
| | 3 | 11.52 | 10.49 | 2.63 |
| | 4 | 13.38 | 12.87 | 2.66 |
| | 5 | 14.29 | 13.34 | 2.56 |
| | 6 | 14.18 | 13.22 | 2.53 |
| | 7 | 14.14 | 13.20 | 0.93 |
| | 8 | 10.86 | 9.78 | 0.93 |
| | 9 | 9.36 | 8.27 | 0.95 |
| | 10 | 8.32 | 7.24 | 0.46 |
| | 24 | 2.09 | 1.88 | 0.25 |

From Table 3, it can be seen that the total saponin content increased with the extraction time and the contents of gypenosides L and LI and ginsenoside Rg3 decreased from 7 hours.

Effect of Extraction Temperature

In order to compare the change in the ingredients and the total saponin content of the Gynostemma pentaphyllum extract depending on extraction temperature, unfermented Gynostemma pentaphyllum was extracted at 85, 95, 102,115, 121 and 130° C. for 4 hours and the contents of detected gypenosides L and LI and ginsenoside Rg3 were compared.

TABLE 4

| | | Gypenoside L | Gypenoside LI | Rg3 |
|---|---|---|---|---|
| Extraction | 85 | 0.23 | 0.12 | 0.22 |
| temperature | 95 | 0.52 | 0.38 | 0.26 |
| (° C.) | 102 | 1.07 | 0.87 | 0.37 |
| | 110 | 3.80 | 2.96 | 0.90 |
| | 115 | 4.21 | 3.18 | 1.11 |
| | 121 | 12.11 | 11.03 | 1.12 |
| | 130 | 5.13 | 4.22 | N.D. |

*N.D.: not detected.

From Table 4, it can be seen that the contents of main saponin ingredients are increased as the extraction temperature is increased to 121° C. It can also be seen that the total saponin content decreases when the extraction temperature exceeds 121° C.

Specifically, the contents of gypenoside L, gypenoside LI and ginsenoside Rg3 in the extract were increased significantly when the extraction temperature was 121° C. as compared to when it was 85° C.

### Test Example 3

### Effect of Fermentation on Saponin Contents

The kinds and contents of saponins in the extracts of Examples 1-3 and Comparative Examples 1-2 were analyzed. As a result of HPLC and Q-TOF analyses, Rb1, Rb2, Rc, Rd, Rg3, Rg5, Rk1, Rh, etc. gypenosides V, IX, XVI, XXXVI, LXXIII, LXVII, LXX, L, LI, etc., gycomoside IV, damulin A, pectolinarin, protopanaxadiol, hydroxysaikosaponin d, majoroside F5, panicolin and canosifloside VI were detected. The contents of main saponin ingredients and total saponins are shown in Table 5.

TABLE 5

| | | | | | | | Comp. | Comp. |
|---|---|---|---|---|---|---|---|---|
| | | Ex. 1 | Ex. 2 | Ex. 3 | Ex. 4 | Ex. 5 | Ex. 1 | Ex. 2 |
| STD | Rg3 | 0.37 | 0.38 | 0.43 | 0.04 | 0.04 | 0.04 | 0.07 |
| | Gp L | 0.16 | 0.15 | 0.17 | 0.15 | 0.14 | <0.01 | 0.02 |
| | Da. A | 0.04 | 0.05 | 0.09 | 0.04 | 0.03 | <0.01 | <0.01 |
| | Rg5 | 0.05 | 0.04 | 0.12 | 0.04 | 0.03 | 0.01 | 0.01 |

| | | | | | | | |
|---|---|---|---|---|---|---|---|
| Total saponins | 1.58 | 1.62 | 1.81 | 1.58 | 1.50 | 0.68 | 0.90 |
| (mg/mL) | | | | | | | |

From Table 5, it can be seen that the contents of total saponins and low-molecular-weight saponins of the extracts of Examples 1-3 according to the present disclosure are significantly increased as compared to those of the comparative examples. In particular, the contents were slightly higher when the fermentation was performed with Aspergillus kawachii.

Meanwhile, dried powder of Gynostemma pentaphyllum was ultrasonicated using an ultrasonic extractor (SEEC-SONIC II, UL-Tech, Korea) under the condition of 25° C., 20 kHz, 750 watt and 20 amplitude before the fermentation. Then a circulating water bath was connected to maintain the temperature constant. When the ultrasonication was performed for less than 1 minute, the total saponin content was hardly increased. When the ultrasonication time exceeded 20 minutes, the kinds of detected saponins were decreased and the total saponin content was decreased, too. When the ultrasonication time was between 5 and 15 minutes, the total saponin content was increased by about 13% and the contents of gypenoside L and gypenoside LI were increased by about 15% as compared to when the ultrasonication was not performed.

### Text Example 4

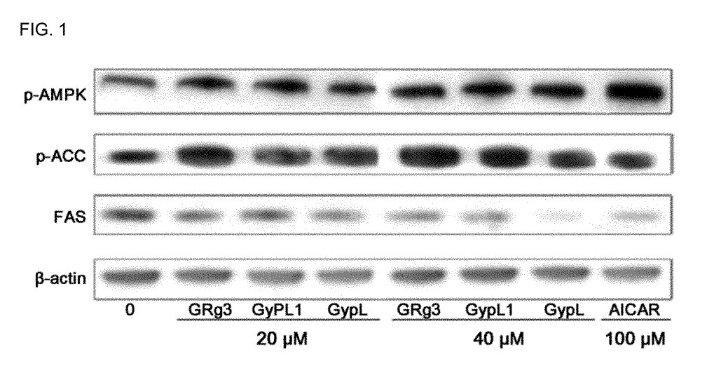

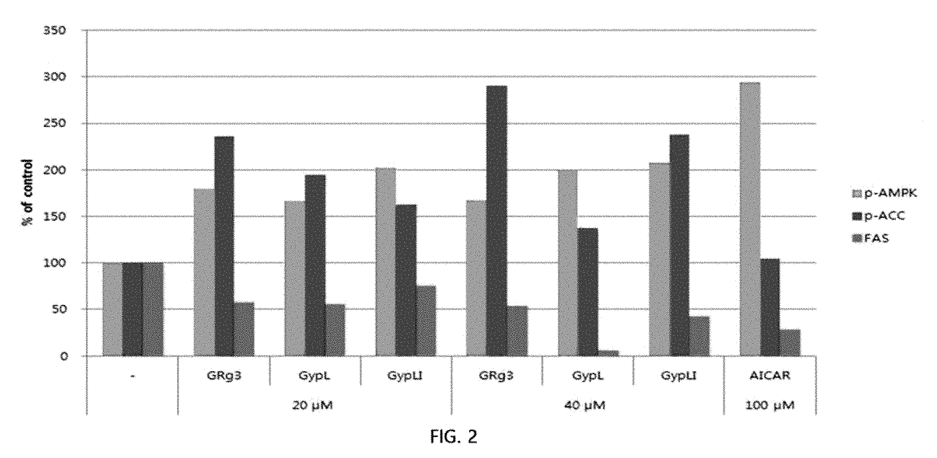

The effect of the saponins of the extract according to the present disclosure on the activity of AMPK, ACC and FAS in 3T3-L1 cells is shown in FIG. 1 and FIG. 2. AMPK is an enzyme involved in fatty acid oxidation, inhibition of adipocyte differentiation, etc. It is involved in inhibition of fatty acid biosynthesis and fatty acid degradation by phosphorylating (inactivating) ACC through AMPK phosphorylation (p-AMPK). FAS (fatty acid synthase) plays a role of promoting fatty acid synthesis.

It was confirmed that the gypenoside LI and ginsenoside Rg3 of the present disclosure activated p-AMPK and p-ACC in a concentration-dependent manner and are effective in inhibiting adipocyte differentiation and fatty acid synthesis by inhibiting the expression of FAS (fatty acid synthase).

### Test Example 5

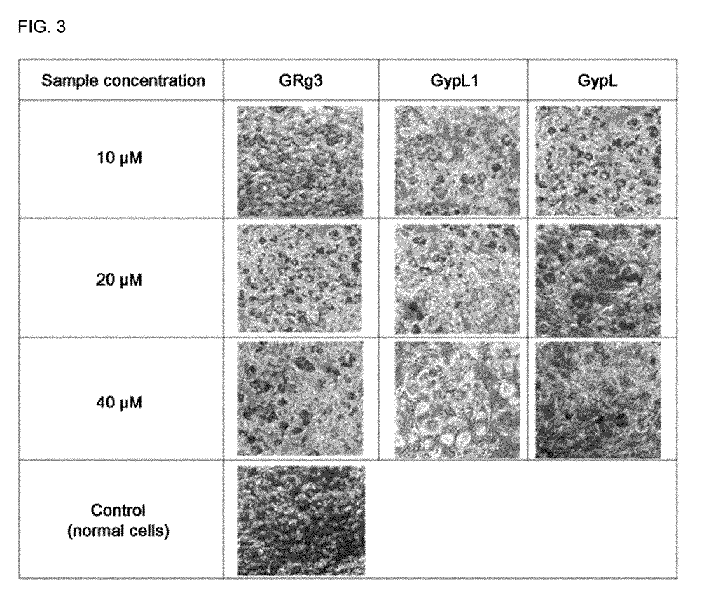

The effect of the saponins of the extract of fermented Gynostemma pentaphyllum according to the present disclosure on fat accumulation was investigated. As seen from FIG. 3, the gypenoside L, gypenoside LI and ginsenoside Rg3 derived from the extract of fermented Gynostemma pentaphyllum according to the present disclosure exhibited an effect of inhibiting fat accumulation as compared to a control group.

1. A method for preparing an extract of fermented Gynostemma pentaphyllum with increased contents of saponins or low-molecular-weight saponins, which comprises:
(1) a step of preparing a Gynostemma pentaphyllum fermentation product by fermenting Gynostemma pentaphyllum with a microorganism selected from Aspergillus oryzae, Aspergillus awamori, Aspergillus kawachii, Lactobacillus brevis and Lactobacillus plantarum; and
(2) a step of extracting the Gynostemma pentaphyllum fermentation product with hot water.

2. The method for preparing an extract of fermented Gynostemma pentaphyllum according to claim 1, wherein the saponin is one or more selected from dammarane-type saponin, gynosaponin, ginsenoside, primeveroside, sweroside, bisdesmoside, gentiobioside and rutinoside.

3. The method for preparing an extract of fermented Gynostemma pentaphyllum according to claim 1, wherein the low-molecular-weight saponin is one or more selected from Rg3, Rg5, Rk1, Rh2, gypenoside L and gypenoside LI.

4. The method for preparing an extract of fermented Gynostemma pentaphyllum according to claim 1, wherein the fermentation is performed at 25-45° C. for 6-240 hours.

5. The method for preparing an extract of fermented Gynostemma pentaphyllum according to claim 1, which further comprises:
a step of preparing an extract of fermented Gynostemma pentaphyllum pomace by extracting fermented Gynostemma pentaphyllum pomace, which is a hot water-extracted Gynostemma pentaphyllum fermentation product, with water or a 20-70% C1-4 alcohol aqueous solution at 30-130° C.; and
a step of mixing the extract of fermented Gynostemma pentaphyllum pomace with the hot water extract of the Gynostemma pentaphyllum fermentation product of the step (1).

6. The method for preparing an extract of fermented Gynostemma pentaphyllum according to claim 1, which further comprises, before fermenting the Gynostemma pentaphyllum, a pretreatment step of ultrasonicating the same with 15-25 kHz and 500-800 watt for 1-20 minutes.

7. A pharmaceutical composition for improving obesity, diabetes or hyperlipidemia, which comprises an extract of fermented Gynostemma pentaphyllum prepared by the method according to claim 4 as an active ingredient.

8. A cosmetic composition for whitening skin or improving wrinkles, which comprises an extract of fermented Gynostemma pentaphyllum prepared by the method according to claim 4 as an active ingredient.

9. A health functional food for restoring stress-induced decline in immune function, which comprises an extract of fermented Gynostemma pentaphyllum prepared by the method according to claim 4 as an active ingredient.

10. An extract of fermented Gynostemma pentaphyllum comprising 5-85 mg/g of gypenoside L and 4-70 mg/g of gypenoside LI, which is a hot water extract of Gynostemma pentaphyllum fermented with a microorganism selected from Aspergillus oryzae, Aspergillus awamori, Aspergillus kawachii, Lactobacillus brevis and Lactobacillus plantarum.
